(19) United States
(12) Patent Application Publication
(10) Pub. No.: US 2015/0367118 A1
(43) Pub. Date: Dec. 24, 2015

(54) HANDHELD DEVICE FOR REPEATED PIERCING OF A HUMAN OR ANIMAL SKIN AND DRIVE MODULE

(71) Applicant: MT.DERM GmbH, Berlin (DE)

(72) Inventors: Dirk Scherkowski, Berlin (DE); Kornelius Knothe, Berlin (DE)

(73) Assignee: MT.DERM GmbH, Berlin (DE)

(21) Appl. No.: 14/736,531

(22) Filed: Jun. 11, 2015

(30) Foreign Application Priority Data
Jun. 12, 2014 (EP) 14 172 106.8

(51) Int. Cl. A61M 37/00

(52) U.S. Cl. CPC A61M 37/0076

(57) ABSTRACT

The application concerns a handheld device for repeated local piercing of a human or animal skin, with a housing, a drive device, a conversion mechanism, and a piercing device, wherein the conversion mechanism has a crank pin (24) that is functionally connected with the drive shaft (21), wherein the crank pin (24) is coupled with a coupling component (25) that can be displaced back and forth transverse to the axial direction of the drive shaft (21), and is connected with the needle reception unit, such that the movement of the crank pin (24) is converted into a forwards and backwards movement of the coupling component (25), and thus of the needle reception unit, transverse to the axial direction of the drive shaft (21). The application furthermore concerns a drive module for a handheld device, together with a method for the operation of a handheld device.

The invention concerns a handheld device for repeated piercing of a human or animal skin, together with a drive module for such a handheld device.

### BACKGROUND

Devices for the local piercing of a human or animal skin are as a rule embodied as handheld devices. Such handheld devices can be used by operating personnel for purposes of applying an ink for a tattoo and/or permanent make-up in the region of the skin surface. However, the introduction of cosmetic or medical agents via the skin is also possible with such devices, in that the skin is locally pierced. Furthermore, such devices can be used without the introduction of any substance, for example so as to stimulate the skin.

A handheld device for local piercing of a skin is, for example, of known art from the publication DE 299 19 199 U1. The handheld device of known art has a handgrip, a drive device and a piercing needle, which in operation is moved with the aid of the drive device forwards and backwards relative to a needle nozzle, wherein at least two modules, detachably connected with one another, are provided, and one of the two modules is designed as a reusable base module with an integrated drive device. The other of the two modules is a sterilised single-use module, in which, in the handheld device of known art, are integrated all components that can be contaminated by the body fluids of a customer. In this manner the handheld device is provided in the form of two modules, of which one, namely the single-use module, can be replaced after use, while the other module, which comprises the drive device, is reused. With the aid of the single-use module the hygienic conditions during the application of a tattoo and/or permanent make-up are improved, since all the parts that can potentially be contaminated, in the treatment of body fluid exiting from the customer, are replaced. Thus the replacement of the whole handheld device is avoided.

From the document EP 1 495 782 A1 a drive module for a device for local piercing of a human or an animal skin is of known art, in which are provided a drive device, with which a drive movement can be generated, and a conversion mechanism coupled to the drive device, with which the drive rotational movement is converted into a forwards/backwards movement, which can be coupled to a piercing device locally piercing the skin, such that a repeated movement of a piercing needle is enabled. The conversion mechanism comprises a functional component, which during the movement conversion executes a tumbling motion or a tilting motion, as a result of which a drive force is provided for purposes of moving a needle locally piercing the skin in a forwards and backwards direction. In one embodiment the functional component is mounted in a freely running manner by means of a ball bearing. An inadvertent rotation of the functional component, which can be brought about as a result of the drive rotational movement, is prevented in the device of known art, in that the functional component mounted by means of a ball bearing, or a projection formed on the functional component, engage in an opening. It has been shown that this form of safeguard for the functional component leads to a not insignificant level of noise pollution when the device is in operation, which in particular is caused by the movement to and fro of the projection in the opening.

The document CN 201 759 989 U discloses a tattooing device with a drive device. The drive device is formed with an eccentric shaft, which provides a drive movement for a needle.

The document U.S. Pat. No. 5,279,552 discloses an intradermal injection device, which is arranged on the skin of a patient, and is used so as to inject, by means of a needle, ink, colouring, or a similar fluid under the skin of a patient.

The document US 2012 / 0279330 A1 relates to a transfer mechanism of an eyebrow tattooing device. The device is formed with an eccentrically rotating shaft, which is coupled with a drive shaft of a motor.

A further tattooing device is disclosed in the document KR 2013 / 0058843 A.

### SUMMARY

The object of the invention is to specify a handheld device for repeated local piercing of a human or animal skin, together with a drive module for this purpose, in which when operating the handheld device the ease of use is improved, in particular with regard to disturbing side effects such as, for example, the noise or vibration of the handheld device.

For purposes of achieving the object a handheld device for repeated piercing of a human or animal skin is created in accordance with the independent Claim 1. Furthermore the independent Claim 12 concerns a drive module for such a handheld device. Advantageous configurations are the subjects of dependent subsidiary claims.

A handheld device for repeated piercing of a human or animal skin is created, which has a housing with a handgrip formed on the latter. When operating the handheld device, which when used for purposes of designing tattoos or permanent make-up can also be also designated as a tattooing device, the user grasps the handgrip in order to guide the handheld device. Other applications provide for the local piercing of the skin for purposes of introducing a cosmetic or a medical agent. However, the handheld device can also be used for local piercing of the human or animal skin without the introduction of any substance, or example for purposes of stimulating the skin. A drive device is accommodated in the housing, with which a rotating drive force is provided via a drive shaft. An electric motor can be provided for this purpose.

A conversion mechanism is coupled to the drive shaft; this mechanism is arranged and constituted in the housing so as to convert the rotating drive movement into a non-rotating drive movement. The drive force provided by this means can, for example, be axially oriented, for example parallel to the axis of the drive shaft. Furthermore a piercing device is provided, which is accommodated in the housing, and has one or a plurality of piercing needles, which are arranged in a needle reception unit. In the case of a plurality of needles these can be arranged in groups in which the needles are located close to one another. However, the distribution of needles or groups of needles over the surface of a needle plate can also be provided. In this case the needle reception unit is formed as a needle plate. The needle reception unit is connected with the conversion mechanism and when in operation is repeatedly moved, together with the one or plurality of piercing needles, forwards and backwards along a movement path in, for example, the axial direction. The movement path can consist of variously designed subsections.

The conversion mechanism has a crank pin inclined to the axial direction of the drive shaft. The crank pin is functionally connected with the drive shaft such that as a result of the rotating drive movement it is displaced into a movement about the axial direction of the drive shaft. In the region of a pick-up point the crank pin is coupled with a coupling component that can be displaced back and forth transverse to the axial direction of the drive shaft, and is connected with the needle reception unit such that the movement of the crank pin is converted into a forwards and backwards movement of the coupling component, and thus of the needle reception unit, transverse to the axial direction of the drive shaft. The forwards and backwards movement of the coupling component can, for example, be executed as a linear movement. When in operation the needle reception unit by this means moves, together with the one or plurality of piercing needles, transverse to the axial direction of the drive shaft backwards and forwards (repeatedly), wherein the tips of the piercing needles can be arranged outside the housing, at least in the end position of the forwards movement.

In the handheld device the drive device, in particular an electric motor, can be accommodated in a section of the housing located transverse to the handgrip on the housing. The drive device can be constituted so as to provide in operation a rotating drive movement of at least some 1,800 rpm (30 Hz).

The housing can consist of a single material, or a combination of various materials, including in particular metals and plastics. Sections of the housing can be detachably mounted, so as to expose regions of the interior of the housing, for example, for purposes of replacement or maintenance. A cable connection can be led out of the housing, serving to provide a connection of the handheld device to an external control unit. The cable connection can, on the one hand, serve to connect an electric motor to a power source. On the other hand the cable connection can have data cables, via which electronic data can be exchanged between the handheld device and the control unit. Such a data exchange between handheld device and control unit can alternatively or additionally also be executed via a wireless data connection, for example using Bluetooth technology. Control units for handheld devices for purposes of repeated local piercing of a human or animal skin are as such, of known art in various forms of embodiment, and are therefore not described in any further detail here.

Furthermore a drive module for a handheld device for repeated local piercing of a human or animal skin is created with a module housing, on which is formed a handgrip. A drive device is accommodated in the module housing, with which a rotating drive movement is provided via a drive shaft. A conversion mechanism is coupled to the drive shaft; this mechanism is arranged and constituted in the module housing so as to convert the rotating drive force into an axially-oriented drive force. On and/or in the module housing is formed a coupling device, which is constituted so as to be detachably coupled to a piercing module, be it, for example, by means of a clipped connection, or a screwed connection. Additionally or alternatively latching elements can be provided. The conversion mechanism has a crank pin functionally connected with the drive shaft and inclined to the axial direction of the drive shaft, which as a result of the rotating drive movement is displaced into a revolutionary type of movement about the drive axis, wherein in the region of a pick-up the crank pin is coupled to a coupling component that can be displaced back and forth transverse to the axial direction of the drive shaft, and is connected with the needle reception unit, such that in operation the movement of the crank pin is converted into a linear forwards and backwards movement of the coupling component transverse to the axial direction of the drive shaft.

In the various forms of embodiment the coupling between conversion mechanism and needle reception unit, which can be formed with a needle shaft, can be embodied such that by virtue of the axially-oriented drive force the needle reception unit is not only driven forwards, but is also pulled backwards, by means of the conversion mechanism comprising the coupling component. In this form of embodiment the conversion mechanism itself provides a restoring force. Alternatively or additionally provision can be made for the restoring force to be provided, at least partially, by an elastic element, for example a spring or a membrane. In this embodiment the outward or forward drive of the needle reception unit is undertaken with the one or plurality of needles against an elastic pre-load, which for its part then contributes to the reverse movement, or effects it on its own. After being stretched during the outward traverse of the piercing needles the elastic element automatically restores its shape and thus effects the reverse movement of the one or plurality of needles.

The crank pin can consist of a hardened material, for example a hardened steel.

The crank pin can be accommodated on a drive dog, which is coupled onto the drive shaft. The drive dog can be embodied as a cylindrical component, which is detachably or non-detachably mounted on the drive shaft. The crank pin can be inclined towards a surface of the drive dog, on which the crank pin is arranged. The surface of the drive dog can be arranged at an angle of some 90° to the drive axis. The drive dog can be embodied as a solid or a hollow component. On an end face facing away from the drive device the crank pin, for example, when embodied as a pin, can be accommodated in an associated recess, either detachably or non-detachably. In the housing of the handheld device the drive dog can be at least partially surrounded by an associated inner housing. In one embodiment the inner housing can be detachably connected with the drive device, for example, by means of a screwed connection. The inner housing can, at the same time, form a protective covering for the drive dog, which rotates when in operation.

The crank pin can be coupled via a ball joint to the coupling component. On the one hand the ball joint allows the revolutionary movement of the crank pin when in operation. On the other hand, on the basis of this movement, it causes the coupling component to be moved forwards and backwards in a linear direction.

The crank pin can be mounted in a freely running manner in an opening on the coupling component. The opening on the coupling component, with which the crank pin engages, can be formed as a slot guide, in which the crank pin runs freely when in operation and which is preferably designed transverse to the axial direction of the drive shaft and to the direction of movement of the coupling component.

The coupling component can be arranged in a guide-oriented transverse to the axial direction of the drive shaft. Some sections of the coupling component, or the whole of the coupling component, can be accommodated in the guide. The overlap between coupling component and associated guide can depend upon the operating positions of the crank pin and the coupling component, wherein the coupling component in a first operating position is arranged completely within the guide, and in a second operating position only partially within the guide. The guide for the coupling component can be embodied as a slot guide.

A stroke adjustment device can be provided, with which a stroke of the forwards and backwards movement of the needle reception unit can be adjusted transverse to the axial direction of the drive shaft. The stroke is the axially-oriented movement between the reversal points of the linear forwards and backwards movement of the needle reception unit. It influences how far the tip of the one or plurality of piercing needles is moved forwards when in operation, whereby in one configuration the piercing depth can be influenced in the local region of the skin that is being pierced. With the aid of the adjustability of the stroke of the axially-oriented drive movement, the handheld device can be adjusted for various applications.

Provision can be made for the stroke to be adjustable while the handheld device is in operation (with the drive device running). To this end the stroke adjustment device can have a stroke adjustment mechanism on the housing, with which it is possible to alter the stroke while the handheld device is in operation, optionally and additionally even if the drive device is not in operation. A rotary sleeve can, for example, be formed on the housing. By rotating the rotary sleeve a stroke adjustment is made possible. Alternatively a sliding component and/or an adjustment lever can be provided on the housing for purposes of adjusting the stroke.

A method can be provided for purposes of operating the handheld device for the repeated local piercing of a human or animal skin, in which, by means of the stroke adjustment device the stroke of the forwards and backwards movement of the needle reception unit transverse to the axial direction of the drive shaft can be adjusted while in operation, that is to say with the drive device running (with rotation of the drive shaft).

With the aid of the stroke adjustment device a distance between a mounting of the foot of the crank pin and the coupling element can be made adjustable. By altering the distance between the mounting of the foot of the crank pin, for example on the drive dog, and the coupling element, the amplitude of the movement introduced into the coupling element is altered, as a result of which the stroke is altered. In general the location or site of the coupling between the crank pin and the coupling component connected to the needle reception unit is altered along the inclined crank pin. By this means the distance between the coupling and the drive shaft is altered in a direction transverse to the axial direction of the drive shaft. The location of the coupling is displaced towards the drive shaft or away from the latter, as a result of which the stroke that is executed is adjusted.

Alternatively or additionally provision can be made for the angle of inclination of the crank pin relative to the axial direction of the drive shaft to be adjustable. To this end provision can be made for the use of crank pins with differing inclinations. Alternatively an inclined crank pin can be provided, which on the component accommodating the crank pin can be adjusted to various inclinations by means of rotation, wherein the crank pin can be latched into the various rotational positions.

The crank pin can be detachably mounted. In one configuration replaceability of the crank pin can be achieved in this manner. Thus, for example, the crank pin can be replaced in the event of wear, or deterioration, optionally also together with the drive dog, wherein the latter option is also possible if the crank pin and drive dog are not connected with one another detachably. Provision can be made for the crank pin arranged in the housing to be accessible via a housing opening, which is closed with a removable housing cover. The housing opening can, for example, be formed on an end face of the housing opposite to the drive device. The housing cover can be embodied in multiple parts, wherein an inner cover of the removable housing cover can have a reception unit for a section of the crank pin. Such a reception unit can be provided on the inner face of the housing cover, even if the housing cover is embodied as a single part.

In one form of embodiment provision can be made for the housing to be formed with multiple housing modules, wherein the drive device is arranged in a drive module, and the piercing device is arranged in a piercing module. The housing modules can be detachably or non-detachably connected with one another. In one configuration in which the housing modules are connected with one another detachably, the piercing module, which can also be designated as the needle module, can be embodied as a sterilised single-use module.

The conversion mechanism can be formed partially in the drive module and partially in the piercing module. Here provision can be made for the coupling component to be arranged in the piercing module, whereas at least the crank pin is accommodated in the drive module. If the drive module and needle module are connected with one another detachably, the crank pin can, for example, when attaching the needle module onto the drive module, be inserted into the associated guide on the coupling component to form the conversion mechanism.

A vibration damping device can be formed on the housing. To this end shock-absorbing particles, for example spherical particles, can be arranged in a cavity on the housing. The particles can consist of tungsten. The particles can be rigidly or flexibly connected with one another. A viscous medium that can flow, or a viscoelastic medium, can surround the particles. Instead of individual particles, or all the particles, a solitary body can also be provided. The cavity can be provided with a closable opening.

A device, at least parts of which are rotating with the drive, can be provided, which, by means of an independently adjustable mass imbalance, effects vibrational balance in the whole of the physical system. Provision can be made for the mass imbalance of the device to be affected by and/or unaffected by the adjustment of the stroke, so that the magnitude of the vibration balance can be matched to the physical system requirements for various stroke adjustments.

The statements made above in connection with the drive module for a handheld device for repeated local piercing of a human or animal skin apply in conjunction with embodiments of the handheld device accordingly.

### DESCRIPTION OF EXAMPLES OF EMBODIMENT

In what follows further examples of embodiment are described in more detail with reference to the figures of a drawing. Here:

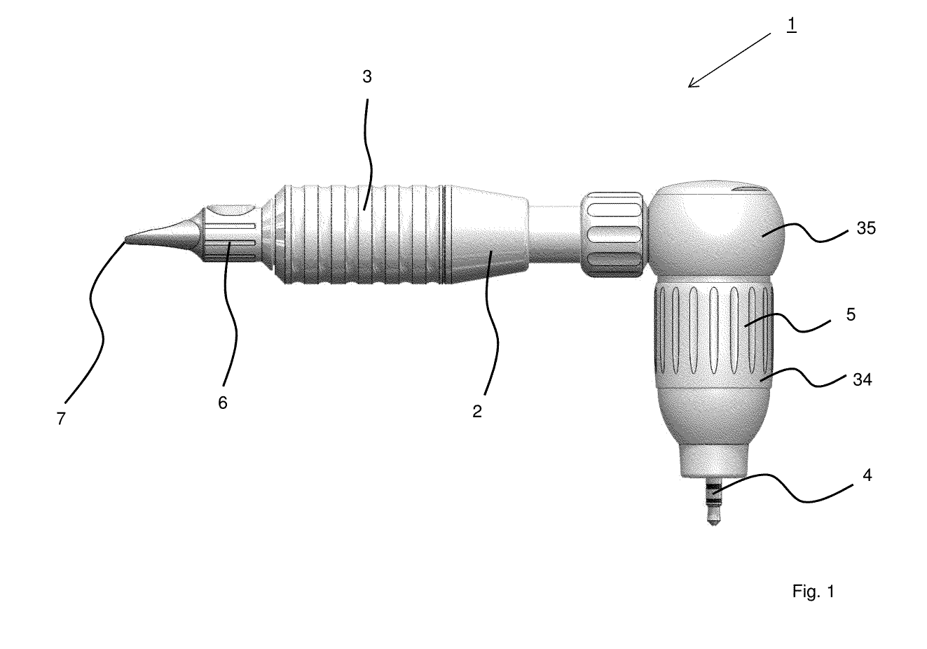

FIG. 1 shows a schematic representation from the side of a handheld device for repeated piercing of a human or animal skin,

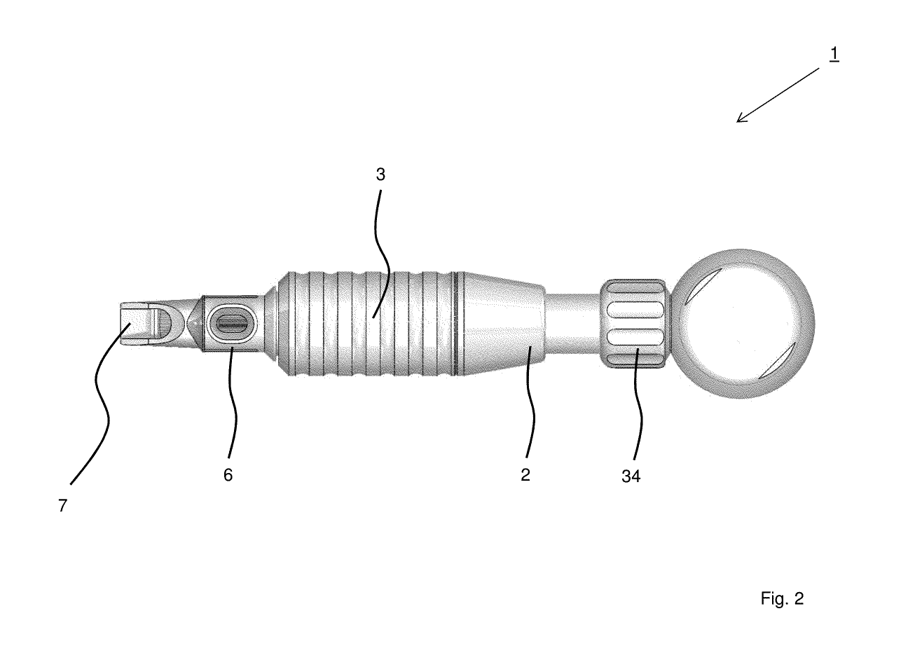

FIG. 2 shows a schematic representation from above of the handheld device from FIG. 1,

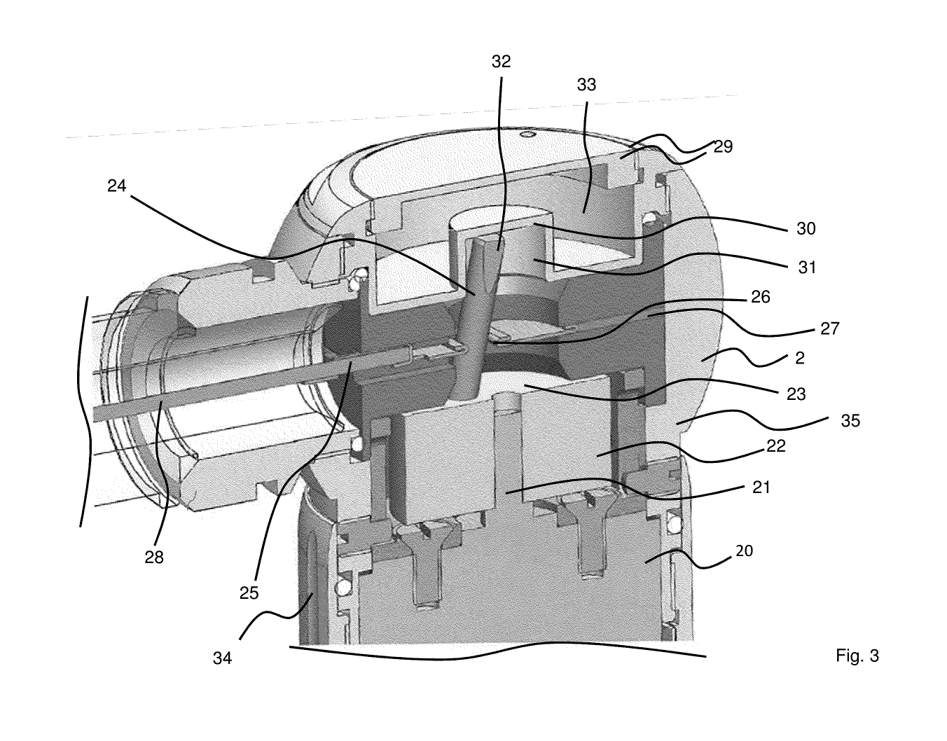

FIG. 3 shows a cross-sectional representation in perspective of a section of the handheld device from FIG. 1,

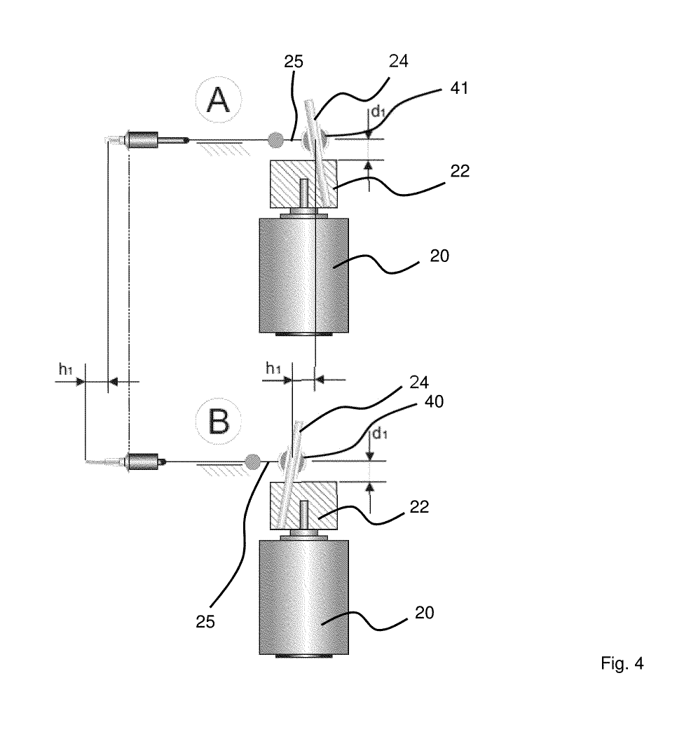

FIG. 4 shows a schematic representation of a drive device with a crank pin in various rotational positions with a stroke h₁, wherein a ball joint decoupling is provided,

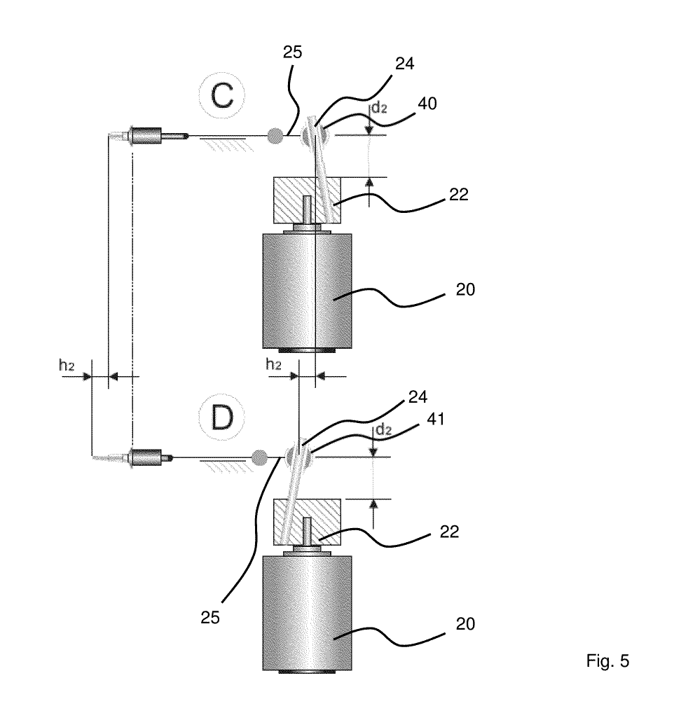

FIG. 5 shows a schematic representation of the drive device with a crank pin from FIG. 4 in various rotational positions, with a stroke h₂<h₁,

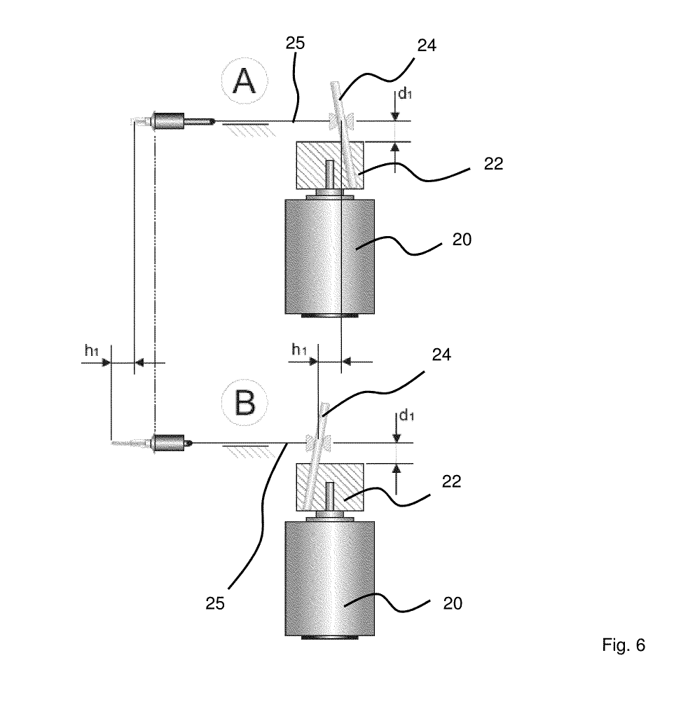

FIG. 6 shows a schematic representation of a drive device with a crank pin in various rotational positions with a stroke h₁, wherein no ball joint decoupling is provided,

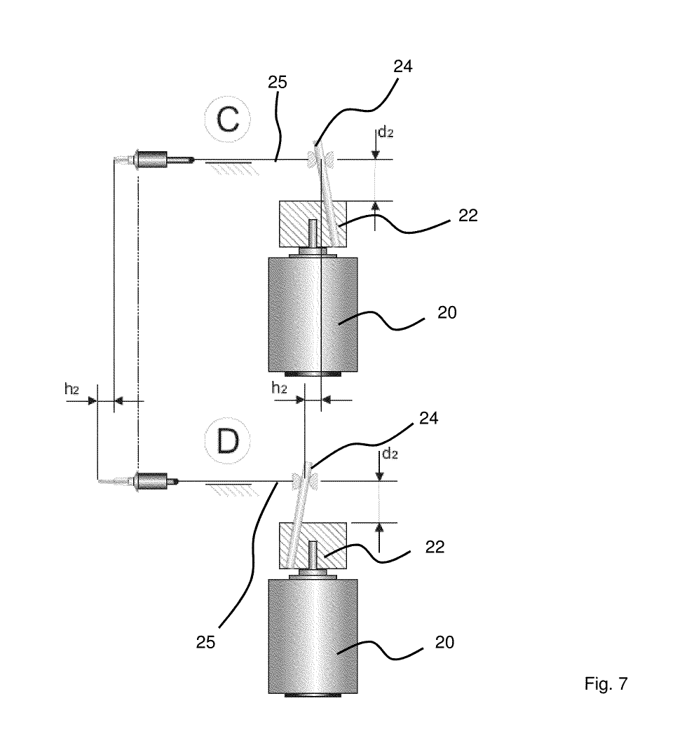

FIG. 7 shows a schematic representation of the drive device with a crank pin from FIG. 6 in various rotational positions, with a stroke h₂<h₁, and

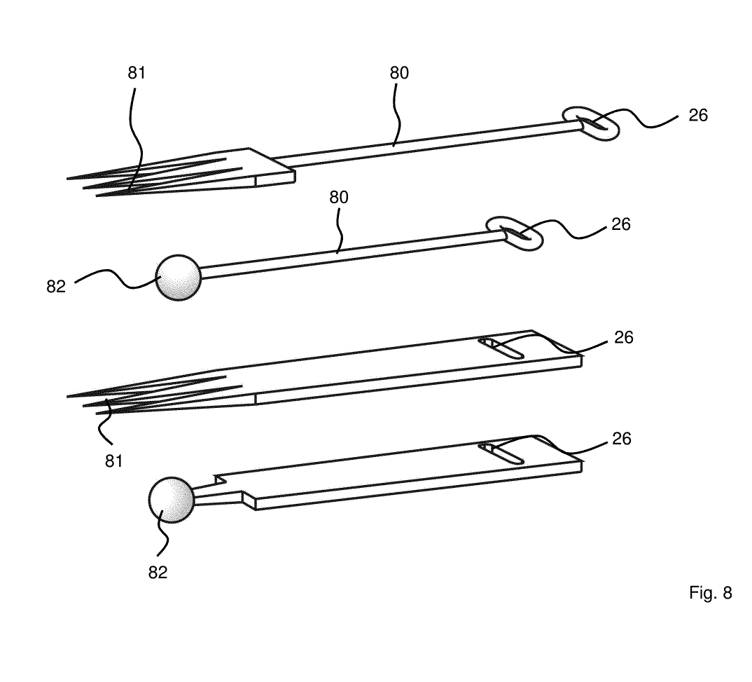

FIG. 8 shows schematic representations of coupling components to be coupled onto an inclined crank pin.

FIG. 1 shows a schematic representation of a handheld device 1 for repeated local piercing of a human or animal skin. A handgrip 3 is externally formed on a housing 2. In the form of embodiment represented an electric motor is incorporated as the drive in the housing 2; this can be connected to a power supply via a plug device 4. Provision can be made for signal or control terminals also to be implemented via the plug device 4, for example for purposes of connecting the handheld device to an external control device (not represented), via which, for example, rotational speed control can be provided for the electric motor.

A drive module 5 is provided, together with a piercing module, or needle module 6. The piercing module 6 can be replaced. In the piercing module 6 one or a plurality of needles are accommodated in the usual manner; in operation these can be traversed out and in through a forward housing opening 7. The one or plurality of needles are for their part arranged in the usual manner in a needle reception unit (not represented).

FIG. 2 shows the handheld device from FIG. 1 from above.

FIG. 3 shows a cross-sectional representation in perspective of a section through the handheld device 1. A connecting or bearing component 22, embodied as a drive dog, is arranged on a drive shaft 21, opposite to an electric motor 20. On an end face 23 of the connecting component 22 is arranged a crank pin 24, which in the form of embodiment represented is embodied as a pin, and is inclined, and for its part engages with an opening in a coupling component 25 mounted such that it can slide; in the operating position shown the pin bears against the opening.

In operation a rotating drive movement of the drive shaft 21 is provided with the aid of the electric motor 20, as a result of which the connecting component 22 rotates, the consequence of which is a movement of the crank pin 24 about the axial direction of the drive shaft 21.

The connecting component 22 and the crank pin 24, also the coupling component 25, are elements of a conversion mechanism, with which the rotational movement provided by the electric motor 20 via the drive shaft 21 is converted into a linear forwards and backwards movement of the coupling component 25. The coupling component 25, in which the opening 26 is arranged in a proximal region, moves when in operation in a guide 27, which here is embodied in the form of a flat slot. As an alternative to the reception of the crank pin 24 in the opening 26 the crank pin 24 can be connected with the coupling component 25 via a ball joint (not represented). Such a connection effects in a similar manner the forwards and backwards movement of the coupling component 25 in the guide 27, if the crank pin 24, as a consequence of the rotating drive movement, moves when in operation about the axial direction of the drive shaft 21. A ball joint-bearing unit can be provided, which implements a plurality of functions such as rotation, sliding, tumbling, and/or gyration at this point.

A distal end 28 of the coupling component 25 couples directly or indirectly onto the needle reception unit (not represented) that is carrying one or a plurality of needles, so that the latter, when in operation, can be moved repeatedly forwards and backwards, in particular along a straight movement path.

In the form of embodiment represented in FIG. 3 a housing cover 29 is arranged on the housing 2 such that it can be removed; on an inner face 30 this has an open reception cavity 30 for a distal end 32 of the crank pin. In operation the distal end 32 moves in the reception cavity 31. Provision can be made for the crank pin 24 to be detachably accommodated on the connecting component 22, for example, to be plugged in, or screwed in. By this means it is made possible in particular to replace the crank pin 24, be it on account of wear, or for purposes of inserting a crank pin with another build shape, for example for purposes of providing an altered inclination of the crank pin 24 to the axial direction of the drive shaft.

Damping agents can be arranged in a cavity 33 in the housing cover 29, for example particles with a spherical shape, in particular tungsten particles, with which vibrations occurring in operation can be damped (shock absorption). The housing cover 29 can be embodied in one piece, or, as shown, in multiple pieces.

A sleeve 34 is accommodated on the housing 2 such that it can rotate. By means of rotation of the sleeve 34, which is coupled, for example, by a thread (not represented) to the component 35, the sleeve 34 is displaced in the axial direction of the drive shaft 21, in particular in a stepless manner, so that by means of relative displacement in the axial direction of the drive shaft 21 the distance between the sleeve 34 and the opening 26 is altered. Here the electric motor 20, together with the connecting or bearing component 22 that is coupled to it, moves with the sleeve, the end effect of which is to alter the location of the opening 26 along the inclined crank pin 24. In the example of embodiment shown this means that the distance between the mounting of the foot of the crank pin 24 and the opening 26 is altered. In this manner the relative position of the opening 26, where the pick-up of the drive movement takes place, along the inclined crank pin 24 is altered, which transverse to the axial direction of the drive shaft 21 alters the distance of the opening 26 (the pickup region) to the drive shaft 21, and thus leads to an adjustment of the stroke.

In this or other embodiments the adjustment of the stroke can also be executed while the drive device is in operation, that is to say, when the drive shaft 21 is rotating.

The stroke adjustment as described by means of displacement of the coupling location (pick-up or pick-up point for the drive movement) along the inclined crank pin 24 can also be deployed in handheld devices that differ from the example of embodiment shown with regard to other design features.

Thus, for example, provision can alternatively or additionally be made for the connecting component 22 to be displaceable in the axial direction on the drive shaft 21, as a result of which the location of the crank pin 24 relative to the opening 26 can be altered for purposes of stroke adjustment. In this context a component (not represented) provided on the housing 2 can enable an external actuation of the displacement, as is also described above for the sleeve 34.

In other words, the crank pin 24, in a manner comparable to a conical shell, executes a rotational movement about the drive shaft 21. An alteration of the distance between the rotation plane and the output plane, which as far as possible lies parallel to the rotation plane, effects a circular motion of greater or lesser size of the inclined crank pin in the output plane about the drive shaft 21. By means of particular pick-up (coupling) options, for example an offset a) of the angle of inclination, or setting angle, and b) of the crank pin rotation per revolution, and/or c) implementation of the sliding capability of the pick-up on the inclined crank pin 24, as far as possible at right angles to the rotation plane, a stroke adjustment is implemented which can be undertaken when in operation, and also independently of the rotational speed.

Here there exists the possibility of offsetting the displacement affecting the needle protrusion, as conditioned by the method, of both dead points (endpoints) of the picked-up linear movement, by altering the stroke such that the forward dead point (=greatest needle protrusion) does not alter, which is very advantageous for the operator of the handheld device.

FIG. 4 shows a schematic representation of a drive device with a crank pin 24 in various rotational positions with a stroke h₁, wherein in the region of a pick-up 40 a ball joint decoupling 41 (ball joint bearing unit) is provided. The representations A and B show different rotational positions for the crank pin 24. An alteration of the distance d of the coupling between crank pin 24 and coupling component 25 alters the stroke h, which in FIG. 5 is shown for a reduced stroke h₂—with an increased distance d₂>d₁ (h₂<h₁). In FIG. 5 the two representations C and D again show different rotational positions for the crank pin 24 (endpoints of the stroke movement).

FIGS. 6 and 7 show in a comparable manner to FIGS. 4 and 5 different rotational positions for the crank pin 24, which is coupled to the coupling component 25 embodied as a flat connecting rod.

FIG. 8 shows various representations of coupling components for purposes of coupling to the crank pin 24. In the upper two configurations the opening 26 is formed in a wire frame 80, whereas in the lower configurations in FIG. 8 a slot is provided as an opening 26. Two of the configurations in FIG. 8 provide a piercing device 81, whereas in the case of the other two configurations a coupling element 82 is formed.

The features of the invention disclosed in the above description, in the claims, together with the figures, can be of importance, both individually and also in any combination, for the implementation of the various embodiments.

1. A handheld device for repeated local piercing of a human or animal skin with:
a housing, on which a handgrip is formed,
a drive device, which is arranged in the housing (2), with which a rotating drive movement is provided via a drive shaft (21),
a conversion mechanism coupling to the drive shaft (21), which mechanism is arranged and constituted in the housing (2) so as to convert the rotating drive movement into a non-rotating drive movement, and
a piercing device, which is arranged in the housing (2), and has one or a plurality of piercing needles, which are arranged in a needle reception unit, which is connected with the conversion mechanism, and which, together with the one or plurality of piercing needles, can be moved repeatedly forwards and backwards along a movement path,
wherein, the conversion mechanism has a crank pin (24) that is functionally connected with the drive shaft (21), which pin is inclined to the axial direction of the drive shaft (21), and as a result of the rotating drive movement is displaced into a movement about the axial direction of the drive shaft (21), wherein the crank pin (24) is coupled in the region of a pick-up (40) with a coupling component (25) that can be displaced back and forth transverse to the axial direction of the drive shaft (21), and is connected with the needle reception unit such that the movement of the crank pin (24) is converted into a forwards and backwards movement of the coupling component (25), and thus of the needle reception unit transverse to the axial direction of the drive shaft (21).

2. The handheld device in accordance with claim 1, wherein the crank pin can be accommodated on a drive dog (22), which is coupled onto the drive shaft (21).

3. The handheld device in accordance with claim 1 or 2, wherein the crank pin (24) is coupled via a ball joint onto the coupling component (25).

4. The handheld device in accordance with claim 1 or 2, wherein the crank pin (24) is mounted in a freely running manner in an opening on the coupling component.

5. The handheld device in accordance with at least one of the preceding claims, wherein the coupling component (25) is arranged in a guide oriented transverse to the axial direction of the drive shaft (21).

6. The handheld device in accordance with at least one of the preceding claims, wherein a stroke adjustment device is provided, with which a stroke of the forwards and backwards movement of the needle reception unit can be adjusted transverse to the axial direction of the drive shaft (21).

7. The handheld device in accordance with claim 6, wherein with the aid of the stroke adjustment device a distance between a mounting of the foot of the crank pin (24) and the coupling component (25) can be adjusted.

8. The handheld device in accordance with at least one of the preceding claims, wherein the crank pin (24) is detachably mounted.

9. The handheld device in accordance with at least one of the preceding claims, wherein the housing (2) is formed with a plurality of housing modules, wherein the drive device is arranged in a drive module and the piercing device is arranged in a piercing module.

10. The handheld device in accordance with claim 9, wherein the conversion mechanism is formed partially in the drive module and partially in the piercing module.

11. The handheld device in accordance with at least one of the preceding claims, wherein a vibration damping device is formed on the housing (2).

12. A drive module for a handheld device for repeated local piercing of a human or animal skin, with:
a module housing, on which a handgrip is formed,
a drive device, which is arranged in the module housing, and with which a rotating drive movement is provided via a drive shaft (21),
a conversion mechanism coupling to the drive shaft (21), which mechanism is arranged and constituted in the module housing so as to convert the rotating drive movement into a non-rotating drive movement, and
a coupling device, which is formed on and/or in the module housing, and is constituted so as to couple detachably to a piercing module,
wherein the conversion mechanism has a crank pin (24) that is functionally connected with the drive shaft (21), which pin is inclined to the axial direction of the drive shaft (21), and as a result of the rotating drive movement is displaced into a movement about an axis parallel to the axial direction of the drive shaft (21), wherein the crank pin (24) is coupled in the region of a pick-up (40) with a coupling component (25) that can be displaced back and forth transverse to the axial direction of the drive shaft (21), and is connected with the needle reception unit such that in operation the movement of the crank pin (24) is converted into a forwards and backwards movement of the coupling component (25) transverse to the axial direction of the drive shaft (21).

13. A method for purposes of operating a handheld device for repeated local piercing of a human or animal skin, in which:
a drive device, which is arranged in a housing (2), provides a rotating drive movement via a drive shaft (21),
a conversion mechanism coupling to the drive shaft (21), which mechanism is arranged in the housing (2), converts the rotating drive movement into a non-rotating drive movement, and
a piercing device, which is arranged in the housing (2), and has one or a plurality of piercing needles, which are arranged in a needle reception unit, is connected with the conversion mechanism, and, together with the one or plurality of piercing needles, is moved repeatedly forwards and backwards along a movement path,
wherein when the drive device is in operation, a stroke of the forwards and backwards movement of the needle reception unit transverse to the axial direction of the drive shaft (21) is adjusted by means of a stroke adjustment device.
